(19) United States
(12) Patent Application Publication
(10) Pub. No.: US 2024/0115482 A1
(43) Pub. Date: Apr. 11, 2024

(54) FORMULATION FOR THE CARE OF DAMAGED HAIR

(71) Applicants: Rohm and Haas Company, Collegeville, PA (US); Union Carbide Corporation, Seadrift, TX (US)

(72) Inventors: Lyndsay M. Leal, Spring City, PA (US); Lu Bai, Novi, MI (US); Emmett M. Partain, III, Bound Brook, NJ (US); Daniel S. Miller, Phoenixville, PA (US); Benjamin Reiner, Collegeville, PA (US)

(21) Appl. No.: 18/263,980

(22) PCT Filed: May 6, 2022
(86) PCT No.: PCT/US2022/027984

§ 371 Date: Aug. 2, 2023

Related U.S. Application Data
(63) Continuation of application No. 63/187,593, filed on May 12, 2021.

(51) Int. Cl. A61K 8/73; A61K 8/891; A61Q 5/02; A61Q 5/12

(52) U.S. Cl. CPC A61K 8/73; A61K 8/891; A61Q 5/02; A61Q 5/12

(57) ABSTRACT

A formulation for the care of damaged hair is provided, comprising: a vehicle; a silicone; and a deposition aid, wherein the deposition aid is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include (i) a quaternary ammonium group of formula (II) bound to a pendent oxygen on the dextran base polymer

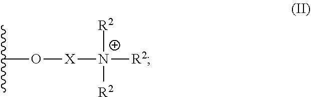

(ii) a quaternary ammonium group of formula (III) bound to a pendent oxygen on the dextran base polymer

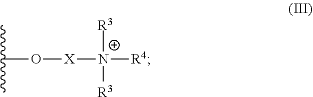

wherein

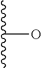 [SMILES: *O]

is pendent oxygen on the dextran base polymer; wherein X is a divalent linking group; wherein each R² is independently selected from a C₁₋₄ alkyl group; wherein each R³ is independently selected from a C₁₋₄ alkyl group; and wherein each R⁴ is independently selected from a C₅₋₂₀ alkyl group.

The present invention relates to a formulation for the care of damaged hair. In particular, the present invention relates to a formulation for the care of damaged hair containing: a dermatologically acceptable vehicle; a dermatologically acceptable silicone; and a deposition aid polymer, wherein the deposition aid polymer is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include (i) a quaternary ammonium group of formula (II) bound to a pendent oxygen on the dextran base polymer

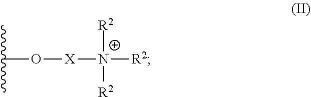

(ii) a quaternary ammonium group of formula (III) bound to a pendent oxygen on the dextran base polymer

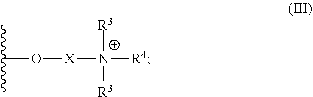

wherein

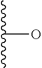 [SMILES: *O]

is a pendent oxygen on the dextran base polymer; wherein X is a divalent linking group; wherein each R² is independently selected from a linear or branched C₁₋₄ alkyl group; wherein each R³ is independently selected from a linear or branched C₁₋₄ alkyl group; and wherein each R⁴ is independently selected from a linear or branched C₅₋₂₀ alkyl group.

Hair cleansing is an ubiquitous component of personal hygiene. Cleansing of hair facilitates the removal of dirt, germs and other things that are perceived as harmful to the hair or the individual. Cleansing formulations typically include a surfactant to promote the removal of materials deposited on the hair. Unfortunately, the cleansing formulations remove both undesirable and desirable materials from hair. For example, cleansing formulations frequently undesirably remove oils from hair; oils operate to protect hair from loss of moisture. Removal of too much oil from hair may leave the hair vulnerable to becoming dry and damaged. One solution to this concern is the selection of mild surfactants. Another approach is to incorporate additives that help replace the oils removed through deposition; however, this approach has proven difficult in implementation, particularly in rinse-off applications.

In addition to shampooing, coloring and styling practices can leave hair with chemical and thermal damage exacerbating the need for conditioning to improve the look and feel of the hair.

In U.S. Pat. No. 7,067,499, Erazo-Majewicz, et al. disclose personal care and household care product composition comprising at least one cationic polygalactomannan or a derivative of cationic polygalactomannans wherein the derivative moiety on the cationic derivatized polygalactomannan is selected from the group consisting of alkyl, hydroxyalkyl, alkylhydroxyalkyl, and carboxymethyl wherein the alkyl has a carbon chain containing from 1 to 22 carbons and the hydroxyalkyl is selected from the group consisting of hydroxyethyl, hydroxypropyl, and hydroxybutyl, wherein the at least one cationic polygalactomannan or derivative of cationic polygalactomannans have a mean average molecular weight (Mw) having a lower limit of 5,000 and an upper limit of 200,000 and having a light transmittance in a 10% aqueous solution of greater than 80% at a light wavelength of 600 nm and a protein content of less than 1.0% by weight of polysaccharide, and aldehyde functionality content of at least 0.01 meq/gram.

While conventionally used deposition aids such as soluble cationic modified celluloses (e.g., polyquaternium-10), guar hydroxypropyltrimonium chloride and other cationic polymers (e.g., polyquatemium-6, polyquaternium-7) provide a certain level of deposition in personal care cleansers; they nevertheless exhibit low efficiency necessitating a relatively high incorporation of the active into the personal care cleanser formulation to facilitate desired results. Such high active (e.g., silicone) levels, however, detrimentally effect the foam/lathery in use consumer feel of the formulation and cost.

Accordingly, there is a continuing need for formulations designed for the care of damaged hair. There is also a continuing need for new benefit agent deposition aids having an increased natural origin index (ISO16128) when compared with conventional benefit agent deposition aids.

The present invention provides a formulation for the care of damaged hair, comprising: a dermatologically acceptable vehicle; a dermatologically acceptable silicone; and a deposition aid polymer, wherein the deposition aid polymer is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include (i) a quaternary ammonium group of formula (II) bound to a pendent oxygen on the dextran base polymer

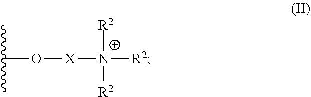

(ii) a quaternary ammonium group of formula (III) bound to a pendent oxygen on the dextran base polymer

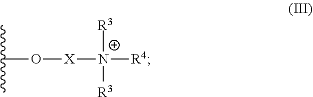

where

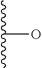 [SMILES: *O]

is a pendent oxygen on the dextran base polymer; wherein X is a divalent linking group; wherein each R² is independently selected from the group consisting of a linear or branched C₁₋₄ alkyl group; wherein each R³ is independently selected from a linear or branched C₁₋₄ alkyl group; and wherein each R⁴ is independently selected from a linear or branched C₅₋₂₀ alkyl group.

The present invention provides a method of caring for damaged hair, comprising: selecting a formulation of the present invention; and applying the formulation to the damaged hair; wherein the deposition aid polymer enhances the deposition of the dermatologically acceptable silicone from the formulation onto the damaged hair relative to an otherwise identical formulation without the deposition aid polymer.

### DETAILED DESCRIPTION

We have surprisingly found that silicone deposition from hair care formulations onto damaged hair can be enhanced through incorporation of a deposition aid polymer, wherein the deposition aid polymer is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include (i) a quaternary ammonium group of formula (II) bound to a pendent oxygen on the dextran base polymer

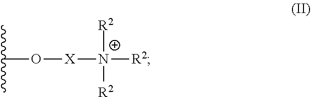

(ii) a quaternary ammonium group of formula (II) bound to a pendent oxygen on the dextran base polymer

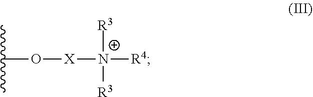

wherein

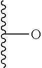 [SMILES: *O]

is a pendent oxygen on the dextran base polymer; wherein X is a divalent linking group; wherein each R² is independently selected from a C₁₋₄ alkyl group; wherein each R³ is independently selected from a C₁₋₄ alkyl group; and wherein each R⁴ is independently selected from a linear or branched C₅₋₂₀ alkyl group.

Unless otherwise indicated, ratios, percentages, parts, and the like are by weight.

As used herein, unless otherwise indicated, the phrase “molecular weight” or Mw refers to the weight average molecular weight as measured in a conventional manner with gel permeation chromatography (GPC) and conventional standards, such as polyethylene glycol standards. GPC techniques are discussed in detail in Modern Size Exclusion Chromatography, W. W. Yau, J. J. Kirkland, D. D. Bly; Wiley-Interscience, 1979, and in A Guide to Materials Characterization and Chemical Analysis, J. P. Sibilia; VCH., 1988, p. 81-84. Molecular weights are reported herein in units of Daltons, or equivalently, g/mol.

The term “dermatologically acceptable” as used herein and in the appended claims refers to ingredients that are typically used for topical application to the skin, and is intended to underscore that materials that are toxic when present in the amounts typically found in skin care compositions are not contemplated as part of the present invention.

Preferably, the formulation for the care of damaged hair is for at least one of chemically damaged hair (e.g., hair damaged from chemical treatments such as dyeing, bleaching, perming), thermally damaged hair (e.g., hair damaged from exposure to heat via ironing, forced drying, styling) and physical damaged hair (e.g., hair damaged from physical abuse such as friction, pulling, curling). More preferably, the formulation for the care of damaged hair is for chemically damaged hair. Most preferably, the formulation for the care of damaged hair is for bleached hair.

Preferably, the formulation for the care of damaged hair (preferably, mammalian hair; more preferably, human hair) of the present invention is selected from the group consisting of a conditioning shampoo formulation, a rinse off conditioner formulation and a leave on conditioner formulation. More preferably, the formulation of the present invention is selected from the group consisting of a conditioning shampoo formulation and a rinse off conditioner formulation. Most preferably, the formulation of the present invention is a conditioning shampoo formulation.

Preferably, the formulation for the care of damaged hair (preferably, mammalian hair; more preferably, human hair) of the present invention, comprises: a dermatologically acceptable vehicle (preferably, wherein the formulation comprises 25 to 99.895 wt % (more preferably, 45 to 99.83 wt %; still more preferably, 79 to 96.65 wt %; most preferably, 84 to 94.4 wt %), based on weight of the formulation, of a dermatologically acceptable vehicle); a dermatologically acceptable silicone (preferably, wherein the formulation comprises 0.1 to 5 wt % (more preferably, 0.15 to 4 wt %; still more preferably, 0.25 to 2 wt %; most preferably, 0.4 to 1.5 wt %), based on weight of the formulation, of the dermatologically acceptable silicone); and a deposition aid polymer (preferably, 0.005 to 5 wt % (more preferably, 0.01 to 2 wt %; still more preferably, 0.1 to 1 wt %; most preferably, 0.2 to 0.5 wt %), based on weight of the formulation, of the deposition aid polymer), wherein the deposition aid polymer is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include (i) a quaternary ammonium group of formula (H) bound to a pendent oxygen on the dextran base polymer

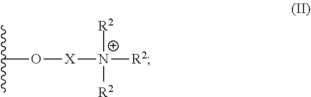

(ii) a quaternary ammonium group of formula (III) bound to a pendent oxygen on the dextran base polymer

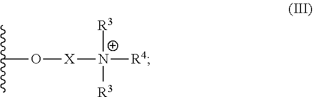

wherein

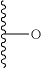 [SMILES: *O]

is a pendent oxygen on the dextran base polymer; wherein X is a divalent linking group; wherein each R² is independently selected from a linear or branched C₁₋₄ alkyl group; wherein each R³ is independently selected from a linear or branched C₁₋₄ alkyl group; and wherein each R⁴ is independently selected from a linear or branched C₅₋₂₀ alkyl group.

Preferably, the formulation for caring of the present invention is a liquid formulation. More preferably, the formulation of the present invention is an aqueous liquid formulation.

Preferably, the formulation for the care of damaged hair (preferably, mammalian hair; more preferably, human hair) of the present invention, comprises: 25 to 99.895 wt % (preferably, 45 to 99.83 wt %; more preferably, 79 to 96.65 wt %; most preferably, 84 to 94.4 wt %), based on weight of the formulation, of a dermatologically acceptable vehicle. More preferably, the formulation for the care of damaged hair (preferably, mammalian hair; more preferably, human hair) of the present invention, comprises: 25 to 99.895 wt % (preferably, 45 to 99.83 wt %; more preferably, 79 to 96.65 wt %; most preferably, 84 to 94.4 wt %), based on weight of the formulation, of a dermatologically acceptable vehicle; wherein the dermatologically acceptable vehicle comprises water. Still more preferably, the formulation for the care of damaged hair (preferably, mammalian hair; more preferably, human hair) of the present invention, comprises: 25 to 99.895 wt % (preferably, 45 to 99.83 wt %; more preferably, 79 to 96.65 wt %; most preferably, 84 to 94.4 wt %), based on weight of the formulation, of a dermatologically acceptable vehicle; wherein the dermatologically acceptable vehicle is selected from the group consisting of water and an aqueous C₁₋₄ alcohol mixture. Most preferably, the formulation for the care of damaged hair (preferably, mammalian hair; more preferably, human hair) of the present invention, comprises: 25 to 99.895 wt % (preferably, 45 to 99.83 wt %; more preferably, 79 to 96.65 wt %; most preferably, 84 to 94.4 wt %), based on weight of the formulation, of a dermatologically acceptable vehicle; wherein the dermatologically acceptable vehicle is water.

Preferably, the water used in the formulation of the present invention is at least one of distilled water and deionized water. More preferably, the water used in the formulation of the present invention is distilled and deionized.

Preferably, the formulation for the care of damaged hair (preferably, mammalian hair; more preferably, human hair) of the present invention, comprises: 0.1 to 5 wt % (preferably, 0.15 to 4 wt %; more preferably, 0.25 to 2 wt %; most preferably, 0.4 to 1.5 wt %), based on weight of the formulation, of a dermatologically acceptable silicone (preferably, wherein the dermatologically acceptable silicone conditions hair). More preferably, the formulation for the care of damaged hair of the present invention, comprises: 0.1 to 5 wt % (preferably, 0.15 to 4 wt %; more preferably, 0.25 to 2 wt %; most preferably, 0.4 to 1.5 wt %), based on weight of the formulation, of a dermatologically acceptable silicone; wherein the dermatologically acceptable silicone is selected from the group consisting of amodimethicone, cyclomethicone, dimethicone, dimethiconol, hexadecyl methicone, hexamethyldisiloxane, diisopropanol amino-PG-propyl disiloxane, methicone, phenyl dimethicone, bis-vinyl dimethicone, stearoxy dimethicone polyalkyl siloxane, polyalkylaryl siloxane, silicone gums (i.e., polydiorganosiloxanes having a weight average molecular weight of 200,000 to 1,000,000 Daltons), polyaminofunctional silicones (e.g., Dow Corning® 929) and combinations thereof. Yet more preferably, the formulation for the care of damaged hair of the present invention, comprises: 0.1 to 5 wt % (preferably, 0.15 to 4 wt %; more preferably, 0.25 to 2 wt %; most preferably, 0.4 to 1.5 wt %), based on weight of the formulation, of a dermatologically acceptable silicone; wherein the dermatologically acceptable silicone is selected from the group consisting of amodimethicone, cyclomethicone, dimethicone, dimethiconol, hexadecyl methicone, hexamethyldisiloxane, methicone, phenyl dimethicone, stearoxy dimethicone and mixtures thereof. Still yet more preferably, the formulation for the care of damaged hair of the present invention, comprises: 0.1 to 5 wt % (preferably, 0.15 to 4 wt %; more preferably, 0.25 to 2 wt %; most preferably, 0.4 to 1.5 wt %), based on weight of the formulation, of a dermatologically acceptable silicone; wherein the dermatologically acceptable silicone is selected from the group consisting of amodimethicone, cyclomethicone, dimethicone, dimethiconol, hexadecyl methicone, methicone and mixtures thereof. Still more preferably, the formulation for the care of damaged hair of the present invention, comprises: 0.1 to 5 wt % (preferably, 0.15 to 4 wt %; more preferably, 0.25 to 2 wt %; most preferably, 0.4 to 1.5 wt %), based on weight of the formulation, of a dermatologically acceptable silicone; wherein the dermatologically acceptable silicone is selected from the group consisting of amodimethicone, dimethicone, dimethiconol and a mixture thereof. Most preferably, the formulation for the care of damaged hair of the present invention, comprises: 0.1 to 5 wt % (preferably, 0.15 to 4 wt %; more preferably, 0.25 to 2 wt %; most preferably, 0.4 to 1.5 wt %), based on weight of the formulation, of a dermatologically acceptable silicone; wherein the dermatologically acceptable silicone comprises a dimethiconol.

Preferably, the formulation for the care of damaged hair (preferably, mammalian hair; more preferably, human hair) of the present invention, comprises: 0.005 to 5 wt % (preferably, 0.01 to 2 wt %; more preferably, 0.1 to 1 wt %; most preferably, 0.2 to 0.5 wt %), based on weight of the formulation, of the deposition aid polymer), based on weight of the formulation, of a deposition aid polymer, wherein the deposition aid polymer is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include (i) a quaternary ammonium group of formula (II) bound to a pendent oxygen on dextran the polymer; and (ii) a quaternary ammonium group of formula (III) bound to a pendent oxygen on dextran the polymer.

Preferably, the dextran base polymer has a weight average molecular weight of 50,000 to 3,000,000 Daltons (preferably, 100,000 to 2,000,000 Daltons; more preferably, 125,000 to 1,000,000 Daltons; still more preferably, 130,000 to 650,000 Daltons; most preferably, 145,000 to 525,000 Daltons). More preferably, the dextran base polymer has a weight average molecular weight of 50,000 to 3,000,000 Daltons (preferably, 100,000 to 2,000,000 Daltons; more preferably, 125,000 to 1,000,000 Daltons; still more preferably, 130,000 to 650,000 Daltons; most preferably, 145,000 to 525,000 Daltons); and the dextran base polymer is a branched chain dextran base polymer comprising a plurality of glucose structural units; wherein 90 to 98 mol % (preferably, 92.5 to 97.5 mol %; more preferably, 93 to 97 mol %; most preferably, 94 to 96 mol %) of the glucose structural units are connected by α-D-1,6 linkages and 2 to 10 mol % (preferably, 2.5 to 7.5 mol %; more preferably, 3 to 7 mol %; most preferably, 4 to 6 mol %) of the glucose structural units are connected by α-1,3 linkages. Most preferably, the dextran base polymer has a weight average molecular weight of 50,000 to 3,000,000 Daltons (preferably, 100,000 to 2,000,000 Daltons; more preferably, 125,000 to 1,000,000 Daltons; still more preferably, 130,000 to 650,000 Daltons; most preferably, 145,000 to 525,000 Daltons); and the dextran base polymer is a branched chain dextran polymer comprising a plurality of glucose structural units; wherein 90 to 98 mol % (preferably, 92.5 to 97.5 mol %; more preferably, 93 to 97 mol %; most preferably, 94 to 96 mol %) of the glucose structural units are connected by α-D-1,6 linkages and 2 to 10 mol % (preferably, 2.5 to 7.5 mol %; more preferably, 3 to 7 mol %; most preferably, 4 to 6 mol %) of the glucose structural units are connected by α-1,3 linkages according to formula I

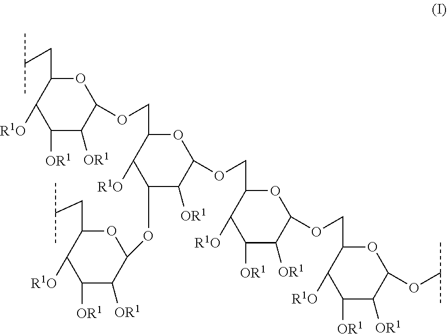

wherein R¹ is selected from a hydrogen, a C₁₋₄ alkyl group and a hydroxy C₁₋₄ alkyl group; and wherein the average branch off the dextran polymer backbone is ≤3 anhydroglucose units.

Preferably, the dextran base polymer contains less than 0.01 wt %, based on weight of the dextran base polymer, of alternan. More preferably, the dextran base polymer contains less than 0.001 wt %, based on weight of the dextran base polymer, of alternan. Most preferably, the dextran base polymer contains less than the detectable limit of alternan.

Preferably, the formulation for the care of damaged hair (preferably, mammalian hair; more preferably, human hair) of the present invention comprises 0.005 to 5 wt % (preferably, 0.01 to 2 wt %; more preferably, 0.1 to 1 wt %; most preferably, 0.2 to 0.5 wt %), based on weight of the formulation, of the deposition aid polymer), based on weight of the formulation, of a deposition aid polymer, wherein the deposition aid polymer is a cationic dextran polymer, comprising a dextran base polymer functionalized with quaternary ammonium groups; wherein the quaternary ammonium groups include (i) a quaternary ammonium group of formula (II) bound to a pendent oxygen on the dextran base polymer

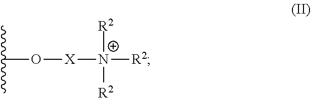

(ii) a quaternary ammonium group of formula (III) bound to a pendent oxygen on the dextran base polymer

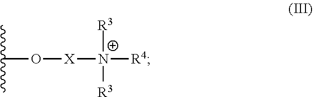

wherein

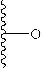 [SMILES: *O]

is a pendent oxygen on the dextran base polymer; wherein X is a divalent linking group; wherein X is a divalent linking group (preferably, wherein X is selected from divalent alkyl groups, which may optionally be substituted with a hydroxy group, an alkoxy group and/or an ether group; more preferably, wherein X is a —CH₂CH(OR⁵)CH₂— group, where R⁵ is selected from the group consisting of a hydrogen and a linear or branched C₁₋₄ alkyl group; most preferably, wherein X is a —CH₂CH(OH)CH₂— group); wherein each R² is independently selected from a linear or branched C₁₋₄ alkyl group (preferably, a linear or branched C₁₋₃ alkyl group; more preferably, a C₁₋₂ alkyl group; most preferably, a methyl group); wherein each R³ is independently selected from a linear or branched C₁₋₄ alkyl group (preferably, a linear or branched C₁₋₃ alkyl group; more preferably, a C₁₋₂ alkyl group; most preferably, a methyl group); and wherein each R⁴ is independently selected from a linear or branched C₅₋₂₀ alkyl group (preferably, a linear or branched C₇₋₁₈ alkyl group; more preferably, a linear or branched C₅₋₂₀ alkyl group; still more preferably, a linear or branched C₁₀₋₁₄ alkyl group; most preferably, a linear or branched C₇₋₁₈ alkyl group). More preferably, the formulation for the care of damaged hair of the present invention comprises 0.005 to 5 wt % (preferably, 0.01 to 2 wt %; more preferably, 0.1 to 1 wt %; most preferably, 0.2 to 0.5 wt %), based on weight of the formulation, of the deposition aid polymer), based on weight of the formulation, of a deposition aid polymer; wherein the deposition aid polymer is a cationic dextran polymer, comprising a dextran base polymer functionalized with quaternary ammonium groups; wherein the quaternary ammonium groups include (i) a quaternary ammonium group of formula (IIa) bound to a pendent oxygen on the dextran base polymer

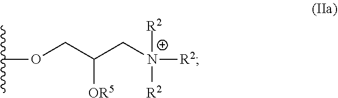

(ii) a quaternary ammonium group of formula (IIIa) bound to a pendent oxygen on the dextran base polymer

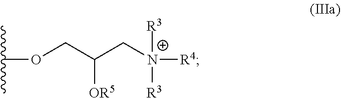

wherein

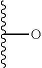 [SMILES: *O]

is a pendent oxygen on the dextran base polymer; wherein each R² is independently selected from a linear or branched C₁₋₄ alkyl group (preferably, a C₁₋₃ alkyl group; more preferably, a C₁₋₂ alkyl group; most preferably, a methyl group); wherein each R³ is independently selected from a linear or branched C₁₋₄ alkyl group (preferably, a C₁₋₃ alkyl group; more preferably, a C₁₋₂ alkyl group; most preferably, a methyl group); wherein each R⁴ is independently selected from a linear or branched C₅₋₂₀ alkyl group (preferably, a linear or branched C₇₋₁₈ alkyl group; more preferably, a linear or branched C₈₋₁₆ alkyl group; still more preferably, a linear or branched C₁₀₋₁₄ alkyl group; most preferably, a linear or branched C₁₂ alkyl group); and wherein each R⁵ is independently selected from the group consisting of a hydrogen and a linear or branched C₁₋₄ alkyl group (preferably, a hydrogen). Most preferably, the formulation for the care of damaged hair of the present invention comprises 0.05 to 5 wt % (preferably, 0.1 to 2 wt %; more preferably, 0.15 to 1 wt %; most preferably, 0.2 to 0.5 wt %), based on weight of the formulation, of a deposition aid polymer; wherein the deposition aid polymer is a cationic dextran polymer, comprising a dextran base polymer functionalized with quaternary ammonium groups; wherein the quaternary ammonium groups include (i) a quaternary ammonium group of formula (IIa) bound to a pendent oxygen on dextran the polymer; and (ii) a quaternary ammonium group of formula (IIIa) bound to a pendent oxygen on dextran the polymer; wherein each R² is a methyl group; wherein each R³ is a methyl group; wherein each R⁴ is independently selected from a linear or branched C₈₋₁₆ alkyl group (preferably, a linear or branched C₁₀₋₁₄ alkyl group; most preferably, a linear or branched C₁₂ alkyl group); and wherein each R⁵ is a hydrogen.

Preferably, the deposition aid polymer has a Kjeldahl nitrogen content, TKN, of 0.5 to 5.0 wt % (preferably, 0.7 to 4 wt %; more preferably, 1 to 3 wt %; most preferably, 1.4 to 2.5 wt %) measured using a Buchi KjelMaster K-375 automated analyzer, corrected for volatiles and ash measured as described in ASTM method D-2364.

Preferably, the deposition aid polymer has a Kjeldahl nitrogen content, TKN, of >1 to 5% measured using a Buchi KjelMaster K-375 automated analyzer, corrected for volatiles and ash measured as described in ASTM method D-2364; wherein the dextran base polymer is functionalized with quaternary ammonium groups; wherein the quaternary ammonium groups include (i) a quaternary ammonium group of formula (II), and (ii) a quaternary ammonium group of formula (III); wherein the deposition aid polymer has a cationic degree of substitution, DS, of dimethyldodecyl ammonium moieties of >0 to 0.03 (preferably, 0.001 to <0.03).

Preferably, the deposition aid polymer comprises <0.001 meq/gram (preferably, <0.0001 meq/gram; more preferably, <0.00001 meq/gram; most preferably, <detectable limit) of aldehyde functionality.

Preferably, the deposition aid polymer comprises <0.1% (preferably, <0.01%; more preferably, <0.001%; most preferably, <detectable limit), of the linkages between individual glucose units in the deposition aid polymer are β-1,4 linkages.

Preferably, the deposition aid polymer comprises <0.1% (preferably, <0.01%; more preferably, <0.001%; most preferably, <detectable limit), of the linkages between individual glucose units in the deposition aid polymer are β-1,3 linkages.

Preferably, the deposition aid polymer comprises <0.001 meq/gram (preferably, <0.0001 meq/gram; more preferably, <0.00001 meq/gram; most preferably, <detectable limit) of silicone containing functionality.

Preferably, the formulation for the care of damaged hair (preferably, mammalian hair; more preferably, human hair) of the present invention, contains <0.01 wt % (preferably, <0.001 wt %; more preferably, <0.0001 wt %; most preferably, <detectable limit), based on weight of the formulation of a dermatologically acceptable non-silicone oil. More preferably, the formulation of the present invention, contains <0.01 wt % (preferably, <0.001 wt %; more preferably, <0.0001 wt %; most preferably, <detectable limit), based on weight of the formulation of a dermatologically acceptable non-silicone oil; wherein the dermatologically acceptable non-silicone oil is selected from the group consisting of hydrocarbon oils (e.g., mineral oil, petroleum jelly, polyisobutene, hydrogenated polyisobutene, hydrogenated polydecene, polyisohexadecane; natural oils (e.g., caprylic and capric triglyceride, sunflower oil, soybean oil, coconut oil, argan oil, olive oil, almond oil); fragrance oils (e.g., limonene) and mixtures thereof.

Preferably, the formulation for the care of damaged hair (preferably, mammalian hair, more preferably, human hair) of the present invention optionally further comprises 0 to 74.895 wt % (preferably, 0.01 to 54.84 wt %; more preferably, 2.5 to 20.65 wt %; most preferably, 5 to 15.4 wt %), based on weight of the formulation, a dermatologically acceptable cleaning surfactant. More preferably, the formulation for the care of damaged hair of the present invention optionally further comprises 0 to 74.895 wt % (preferably, 0.01 to 54.84 wt %; more preferably, 2.5 to 20.65 wt %; most preferably, 5 to 15.4 wt %), based on weight of the formulation, of a dermatologically acceptable cleaning surfactant. Still more preferably, the formulation for the care of damaged hair of the present invention optionally further comprises 0 to 74.895 wt % (preferably, 0.01 to 54.84 wt %; more preferably, 2.5 to 20.65 wt %; most preferably, 5 to 15.4 wt %), based on weight of the formulation, of a dermatologically acceptable cleaning surfactant; wherein the dermatologically acceptable cleaning surfactant is selected from the group consisting of alkyl polyglucosides (e.g., lauryl glucoside, coco-glucoside, decyl glucoside), glycinates (e.g., sodium cocoyl glycinate), betaines (e.g., alkyl betaines such as cetyl betaine and amido betaines such as cocamidopropyl betaine), taurates (e.g., sodium methyl cocoyl taurate), glutamates (e.g., sodium cocoyl glutamate), sarcosinates (e.g., sodium lauroyl sarcosinate), isethionates (e.g., sodium cocoyl isethionate, sodium lauroyl methyl isethionate), sulfoacetates (e.g., sodium lauryl sulfoacetate), alaninates (e.g., sodium cocoyl alaninate), amphoacetates (e.g., sodium cocoamphoacetate), sulfates (e.g., sodium lauryl ether sulfate (SLES)), sulfonates (e.g., sodium C₁₄₋₁₆ olefin sulfonate), succinates (e.g., disodium lauryl sulfosuccinate), fatty alkanolamides (e.g., cocamide monoethanolamine, cocamide diethanolamine, soyamide diethanolamine, lauramide diethanolamine, oleamide monoisopropanolamine, stearamide monoethanolamine, myristamide monoethanolamine, lauramide monoethanolamine, capramide diethanolamine, ricinoleamide diethanolamine, myristamide diethanolamine, stearamide diethanolamine, oleylamide diethanolamine, tallowamide diethanolamine, lauramide monoisopropanolamine, tallowamide monoethanolamine, isostearamide diethanolamine, isostearamide monoethanolamine) and mixtures thereof. Yet more preferably, the formulation for the care of damaged hair of the present invention optionally further comprises 0 to 74.895 wt % (preferably, 0.01 to 54.84 wt %; more preferably, 2.5 to 20.65 wt %; most preferably, 5 to 15.4 wt %), based on weight of the formulation, of a dermatologically acceptable cleaning surfactant; wherein the dermatologically acceptable cleaning surfactant includes a sodium lauryl ether sulfate. Most preferably, the formulation for the care of damaged hair of the present invention optionally further comprises 0 to 74.895 wt % (preferably, 0.01 to 54.84 wt %; more preferably, 2.5 to 20.65 wt %; most preferably, 5 to 15.4 wt %), based on weight of the formulation, of a dermatologically acceptable cleaning surfactant; wherein the dermatologically acceptable cleaning surfactant includes a blend of a sodium lauryl ether sulfate, a cocamide monoethanolamine and a cocamidopropyl betaine.

Preferably, the formulation for the care of damaged hair (preferably, mammalian hair; more preferably, human hair) of the present invention, optionally, further comprises at least one additional ingredient selected from the group consisting of an antimicrobial agent/preservative (e.g., benzoic acid, sorbic acid, phenoxyethanol, methylisothiazolinone, ethylhexyl glycerin); a rheology modifier (e.g., PEG-150 pentaerythrityl tetrastearate); a soap; a colorant; pH adjusting agent; an antioxidant (e.g., butylated hydroxytoluene); a humectant (e.g., glycerin, sorbitol, monoglycerides, lecithins, glycolipids, fatty alcohols, fatty acids, polysaccharides, sorbitan esters, polysorbates (e.g., Polysorbate 20, Polysorbate 40, Polysorbate 60, and Polysorbate 80), diols (e.g., propylene glycol), diol analogs, triols, triol analogs, cationic polymeric polyols); a wax; a foaming agent; an emulsifying agent; a colorant; a fragrance; a chelating agent (e.g., tetrasodium ethylene diamine tetraacetic acid); a preservative (e.g., benzoic acid, sorbic acid, phenoxyethanol, methylisothiazolinone); a bleaching agent; a lubricating agent; a sensory modifier, a sunscreen additive; a vitamin; a protein/amino acid; a plant extract; a natural ingredient; a bioactive agent; an anti-aging agent; a pigment; an acid; a penetrant; an anti-static agent; an anti-frizz agent; an antidandruff agent; a hair waving/straightening agent; a hair styling agent; a hair oil; natural oils or ester emollients (e.g., mono-, di-, tri-glycerides such as sunflower seed oil, coconut oil, cottonseed oil, borage oil, borage seed oil, primrose oil, castor and hydrogenated castor oils, rice bran oil, soybean oil, olive oil, safflower oil, shea butter, jojoba oil and combinations thereof); an absorbent; a hard particle; a soft particle; a conditioning agent (e.g., guar hydroxypropyltrimonium chloride, PQ-10, PQ-7); a slip agent; an opacifier, a pearlizing agent and a salt. More preferably, the formulation for the care of damaged hair of the present invention, optionally, further comprises at least one additional ingredient selected from the group consisting of an antimicrobial agent/preservative (e.g., benzoic acid, sorbic acid, phenoxyethanol, methylisothiazolinone, ethylhexyl glycerin); a rheology modifier (e.g., PEG-150 pentaerythrityl tetrastearate); and a chelating agent (e.g., tetrasodium ethylene diamine tetraacetic acid). Most preferably, the formulation for the care of damaged hair of the present invention, optionally, further comprises at least one additional ingredient selected from the group consisting of a mixture of phenoxyethanol and methylisothiazolinone; a mixture of phenoxyethanol and ethylhexyl glycerin; PEG-150 pentaerythrityl tetrastearate; and tetrasodium ethylene diamine tetraacetic acid.

Preferably, the formulation for the care of damaged hair (preferably, mammalian hair, more preferably, human hair) of the present invention further comprises a thickener. More preferably, the formulation for the care of damage hair of the present invention further comprises a thickener, wherein the thickener is selected to increase the viscosity of the formulation, preferably without substantially modifying the other properties of the formulation. Preferably, the formulation for the care of damaged hair of the present invention further comprises a thickener, wherein the thickener is selected to increase the viscosity of the formulation, preferably without substantially modifying the other properties of the formulation and wherein the thickener accounts for 0 to 5.0 wt % (preferably, 0.1 to 5.0 wt %; more preferably, 0.2 to 2.5 wt %; most preferably, 0.5 to 2.0 wt %), based on weight of the formulation.

Preferably, the formulation for the care of damaged hair (preferably, mammalian hair; more preferably, human hair) of the present invention further comprises an antimicrobial agent/preservative. More preferably, the formulation for the care of damaged hair of the present invention further comprises an antimicrobial/preservative, wherein the antimicrobial/preservative is selected from the group consisting of phenoxyethanol, ethylhexyl glycerin, benzoic acid, benzyl alcohol, sodium benzoate, DMDM hydantoin, 2-ethylhexyl glyceryl ether, isothiazolinone (e.g., methylchloroisothiazolinone, methylisothiazolinone) and mixtures thereof. Most preferably, the formulation for the care of damaged hair of the present invention, further comprises an antimicrobial/preservative, wherein the antimicrobial/preservative is a mixture selected from the group consisting of (a) phenoxyethanol and ethylhexyl glycerin and (b) phenoxyethanol and an isothiazolinone (more preferably, wherein the antimicrobial/preservative is a mixture selected from the group consisting of (a) phenoxyethanol and ethylhexyl glycerin and (b) phenoxyethanol and methylisothiazolinone; most preferably, wherein the antimicrobial/preservative is a mixture of phenoxyethanol and ethylhexyl glycerin).

Preferably, the formulation for the care of damaged hair (preferably, mammalian hair; more preferably, human hair) of the present invention optionally further comprises a pH adjusting agent. More preferably, the formulation for the care of damaged hair of the present invention, further comprises a pH adjusting agent, wherein the formulation has a pH of 4 to 9 (preferably, 4.25 to 8; more preferably, 4.5 to 7; most preferably, 4.75 to 6).

Preferably, the pH adjusting agent is selected from the group consisting of at least one of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of at least one of citric acid, lactic acid, sodium hydroxide, potassium hydroxide, triethanolamine, amino-2-methyl-1-propanol. Still more preferably, the pH adjusting agent includes citric acid. Most preferably, the pH adjusting agent is citric acid.

Preferably, the method of caring for damaged hair (preferably, mammalian hair; more preferably, human hair) of the present invention, comprises: selecting a formulation of the present invention (preferably, wherein the selected formulation includes a dermatologically acceptable cleaning surfactant) and applying the formulation to the damaged hair, wherein the deposition aid polymer enhances the deposition of the dermatologically acceptable silicone from the formulation onto the damaged hair relative to an otherwise identical formulation without the deposition aid polymer. More preferably, the method of caring for damaged hair (preferably, mammalian hair; more preferably, human hair) of the present invention, comprises: selecting a formulation of the present invention (preferably, wherein the selected formulation includes a dermatologically acceptable cleaning surfactant); wetting the damaged hair with water, applying the formulation to the wetted damaged hair; wherein the deposition aid polymer enhances the deposition of the dermatologically acceptable silicone from the formulation onto the damaged hair relative to an otherwise identical formulation without the deposition aid polymer. Most preferably, the method of caring for damaged hair (preferably, mammalian hair; more preferably, human hair) of the present invention, comprises: selecting a formulation of the present invention (preferably, wherein the selected formulation includes a dermatologically acceptable cleaning surfactant); wetting the damaged hair with water, applying the formulation to the wetted damaged hair; and then rinsing the hair with water; wherein the deposition aid polymer enhances the deposition of the dermatologically acceptable silicone from the formulation onto the damaged hair relative to an otherwise identical formulation without the deposition aid polymer.

Some embodiments of the present invention will now be described in detail in the following Examples.

### Synthesis S1: Synthesis of Cationic Dextran Polymer

A 500 mL, four necked, round bottom flask fitted with a rubber serum cap, a nitrogen inlet, a pressure equalizing addition funnel, a stirring paddle and motor, a subsurface thermocouple connected to a J-KEM controller and a Friedrich condenser connected to a mineral oil bubbler was charged with dextran polymer (30.64 g; Polydex, 150 kDa Mw) and deionized water (160.34 g). The addition funnel was charged with a 70% aqueous solution of 2,3-epoxypropyltrimethylammonium chloride (27.06 g; QUAB® 151 available from SKW QUAB Chemicals). The flask contents were allowed to stir until the dextran polymer dissolved in the deionized water. While the contents were stirring, the apparatus was purged with nitrogen to displace any oxygen entrained in the system. The nitrogen flow rate was about 1 bubble per second. The mixture was purged with nitrogen while stirring for one hour. Using a plastic syringe, a 25% aqueous sodium hydroxide solution (4.75 g) was added over a period of a few minutes to the flask contents with stirring under nitrogen. The flask contents were then allowed to stir under nitrogen for 30 minutes. The contents of the addition funnel were then charged to the flask contents dropwise over a few minutes under nitrogen with continued stirring. After the contents of the addition funnel were transferred to the flask contents, the mixture was allowed to stir for 5 minutes. Then heat was applied to the flask contents with a heating mantle controlled using the J-KEM controller set at 55° C. The flask contents were heated to and maintained at 55° C. for 90 minutes. The flask contents were then cooled to room temperature while maintaining a positive nitrogen pressure in the flask. When the flask contents reached room temperature, glacial acetic acid (2.5 g) was added. The polymer was recovered by non-solvent precipitation from methanol, recovering the precipitated polymer by vacuum filtration using a Buchner funnel and dried overnight in vacuo at 50° C. The product branched chain cationic dextran polymer was an off-white solid (23.9 g), with a volatiles content of 3.54%, an ash content of 0.11% (as sodium chloride). The volatiles and ash were measured as described in ASTM method D-2364. The Kjeldahl nitrogen content, TKN, was measured using a Buchi KjelMaster K-375 automated analyzer, and was found to be 1.053% (corrected for volatiles and ash), which corresponds to a trimethylammonium degree of substitution of 0.138.

### Syntheses S2-S4: Synthesis of Cationic Dextran Polymer

In Syntheses S2-S4, cationic dextran polymers were prepared substantially as described in Synthesis S1 but with the reagents and amounts noted in TABLE 1. The degree of cationic substitution, CS, of the QUAB® 151 in the product cationic dextran polymers measured by NMR is reported in TABLE 3. The total Kjeldahl nitrogen, TKN, in the product cationic dextran polymers is also reported in TABLE 3.

TABLE 1

Dextran Base Polymer

| Example | Charge (g) | Type | QUAB ® 151 (g) |
|---|---|---|---|
| Synthesis S1 | 30.64 | A | 27.06 |
| Synthesis S2 | 27.89 | B | 27.13 |
| Synthesis S3 | 28.13 | B | 27.14 |
| Synthesis S4 | 30.53 | C | 27.09 |

A - Polydex 150 aqueous dextran
B - Sigma Aldrich Catalog number D4876
C - Polydex 250 aqueous dextran

### Synthesis S5: Synthesis of Cationic Dextran Polymer

A 500 mL, four necked, round bottom flask fitted with a rubber serum cap, a nitrogen inlet, a pressure equalizing addition funnel, a stirring paddle and motor, a subsurface thermocouple connected to a J-KEM controller and a Friedrich condenser connected to a mineral oil bubbler was charged with dextran base polymer (125.97 g; 21.4% Polydex aqueous dextran), N,N-dimethyloctylamine (9.83 g) and epichlorohydrin (5.69 g). The contents of the flask were stirred at 70 rpm. While stirring, the head space in the flask was purged with a slow, steady flow of nitrogen (about one bubble per second) for one hour to remove any entrained oxygen in the apparatus.

After the one hour nitrogen purge, heat was applied to the flask contents using a heating mantle and the J-KEM controller (set-point of 70° C.). While stirring under nitrogen, the flask contents were maintained at 70° C. for 5 hours. During this time, the color of the flask contents changed from yellow to dark brown, and the viscosity noticeably increased as the reaction progressed.

The flask contents were then cooled in a water bath while maintaining a positive nitrogen pressure in the flask. A solid polymer product was recovered from the flask contents by non-solvent precipitation with acetone. A Waring blender was charged with 500 mL of acetone and approximately 20 mL of polymer solution was slowly and continuously added at moderate mixing speed using a plastic disposable syringe. The polymer was recovered by vacuum filtration through a Buchner funnel with a fine frit. The Waring blender was charged with fresh acetone and the non-solvent precipitation of the remaining aqueous solution was continued. The polymer was briefly air dried, then dried overnight in vacuo at 50° C. The dried polymer was manually ground with a mortar and pestle, and screened through a US standard #30 sieve.

The product polymer was obtained as a white solid (29.63 g), with a volatiles content of 2.47%, and ash content (as sodium chloride) of 1.84%, and a Kjeldahl nitrogen content (corrected for ash and volatiles) of 1.442%, corresponding to a CS value of 0.225.

### Synthesis S6: Synthesis of Cationic Dextran Polymer

A 500 mL, four necked, round bottom flask fitted with a rubber serum cap, a nitrogen inlet, a pressure equalizing addition funnel, a stirring paddle and motor, a subsurface thermocouple connected to a J-KEM controller and a Friedrich condenser connected to a mineral oil bubbler was charged with dextran base polymer (126.92 g, 21.4% Polydex aqueous dextran), N,N-dimethyldodecylamine (13.54 g) and epichlorohydrin (5.84 g). The contents of the flask were stirred at 70 rpm. While stirring, the head space in the flask was purged with a slow, steady flow of nitrogen (about one bubble per second) for one hour to remove any entrained oxygen in the apparatus.

After the one hour nitrogen purge, heat was applied to the flask contents using a heating mantle and the J-KEM controller (set-point of 70° C.). While stirring under nitrogen, the flask contents were maintained at 70° C. for 5 hours. During this time, the color of the flask contents changed from yellow to dark brown, and the viscosity noticeably increased as the reaction progressed.

The flask contents were then cooled in a water bath while maintaining a positive nitrogen pressure in the flask. A solid polymer product was recovered from the flask contents by non-solvent precipitation with acetone. A Waring blender was charged with 500 mL of acetone and approximately 20 mL of polymer solution was slowly and continuously added at moderate mixing speed using a plastic disposable syringe. The polymer was recovered by vacuum filtration through a Buchner funnel with a fine frit. The Waring blender was charged with fresh acetone and the non-solvent precipitation of the remaining aqueous solution was continued. The polymer was briefly air dried, then dried overnight in vacuo at 50° C. The dried polymer was manually ground with a mortar and pestle, and screened through a US standard #30 sieve.

The product polymer was obtained as a white solid (29.96 g), with a volatiles content of 2.35%, and ash content (as sodium chloride) of 1.99%, and a Kjeldahl nitrogen content (corrected for ash and volatiles) of 1.079%, corresponding to a CS value of 0.163.

### Synthesis S7: Synthesis of Cationic Dextran Polymer

A 500 mL, four necked, round bottom flask fitted with a rubber serum cap, a nitrogen inlet, a pressure equalizing addition funnel, a stirring paddle and motor, a subsurface thermocouple connected to a J-KEM controller and a Friedrich condenser connected to a mineral oil bubbler was charged with dextran base polymer (25.0 g; Sigma Aldrich catalog #D4876) and deionized water (100 g). The contents of the flask were stirred at 70 rpm. While stirring, the head space in the flask was purged with a slow, steady flow of nitrogen (about one bubble per second) for one hour to remove any entrained oxygen in the apparatus.

The addition funnel was charged with a 70% aqueous solution of 2,3-epoxypropyltrimethylammonium chloride (30.0 g; QUAB® 151 available from SKW QUAB Chemicals) and a 40% aqueous solution of 3-chloro-2-hydroxypropyl-lauryl-dimethylammonium chloride (39.3 g; QUAB® 342 available from SKW QUAB Chemicals).

While stirring the flask contents under nitrogen, a 25% aqueous sodium hydroxide solution (11 g) was added to the flask contents over 2 minutes. The flask contents were then continually stirred for one hour before adding the contents of the addition funnel to the flask contents dropwise over 3 minutes. The flask contents were then stirred for 20 minutes before heating the flask contents using a heating mantle with a set point temperature of 55° C. for 1.5 hours. The flask contents were then cooled in an ice water bath while maintaining a positive nitrogen pressure in the flask. The flask contents were then neutralized by adding glacial acetic acid (1.66 g) to the flask contents. The flask contents were then stirred for 10 minutes under nitrogen. A polymer product was then recovered from the flask contents by non-solvent precipitation in methanol; roughly 1 L of methanol was used. The methanol was then decanted off and the polymer product was placed in a dish and dried in vacuo at 50° C. overnight.

The polymer product recovered was sieved through a 30 mesh screen and obtained as a free-flowing white solid (27.2 g) with a volatiles content of 5.13% and an ash content (as sodium acetate) of 0.56%. The total Kjeldahl nitrogen in the polymer product was determined to be 1.329 wt %.

### Syntheses S8-S10: Synthesis of Cationic Dextran Polymer

In Syntheses S8-10, cationic dextran polymers were prepared substantially as described in Synthesis S7 but with varying reagent feeds as noted in TABLE 2. The degree of cationic substitution, CS, of the QUAB® 151 and of the QUAB® 342 in the product cationic dextran polymers measured by NMR is reported in TABLE 3. The total Kjeldahl nitrogen, TKN, in the product cationic dextran polymers is also reported in TABLE 3.

TABLE 2

| | | | QUAB ® | QUAB ® |
|---|---|---|---|---|
| Example | Dextran (g) | NaOH (g) | 151 (g) | 342 (g) |
| Synthesis S7 | 25.0 | 11.4 | 30.1 | 39.4 |
| Synthesis S8 | 25.0 | 7.5 | 30.1 | 19.7 |
| Synthesis S9 | 25.1 | 5.6 | 30.0 | 9.9 |
| Synthesis S10 | 25.1 | 4.6 | 30.0 | 4.9 |

TABLE 3

| | Degree of cationic substitution, CS | | | | TKN |
|---|---|---|---|---|---|
| Ex. | QUAB ® 151 | N,N-dimethyloctylamine | QUAB ® 342 | Total | (wt %) |
| Syn. S1 | 0.138 | 0 | 0 | 0.138 | 1.053 |
| Syn. S2 | 0.192 | 0 | 0 | 0.192 | 1.406 |
| Syn. S3 | 0.165 | 0 | 0 | 0.165 | 1.233 |
| Syn. S4 | 0.146 | 0 | 0 | 0.146 | 1.112 |
| Syn. S5 | 0 | 0.225 | 0 | 0.225 | 1.442 |
| Syn. S6 | 0 | 0 | 0.163 | 0.163 | 1.079 |
| Syn. S7 | 0.121 | 0 | 0.013 | 0.134 | 1.329 |
| Syn. S8 | 0.191 | 0 | 0.008 | 0.199 | 1.461 |
| Syn. S9 | 0.164 | 0 | 0.007 | 0.171 | 1.594 |
| Syn. S10 | 0.141 | 0 | 0.005 | 0.146 | 1.706 |

### Comparative Examples CF1-CF7 and Examples F1-F4: Shampoo Formulations

A shampoo formulation was prepared in each of Comparative Examples CF1-CF7 and Examples F1-F7 having the formulation noted in TABLE 4. Specifically, the shampoo formulations were prepared in each of Comparative Examples CF1-CF7 and Examples F1-F4 using the following process: In a container, the 30 wt % aqueous solution of sodium lauryl sulfate was dissolved in 20 g of deionized water and heated to 70° C. with constant stirring. The polymer noted in TABLE 4 was then added to the container with stirring (e.g., guar hydroxypropyltrimonium chloride, cationic dextran prepared according to one of Syntheses S1-S10). When the polymer was dissolved, the tetrasodium EDTA was then added to the container. Once the container contents reached 70° C., the 45 wt % aqueous solution a portion of the PEG-150 pentaerythrityl tetrastearate and the 30 wt % aqueous solution of cocamide MEA were added to the container. Then the 30 wt % solution of cocamidopropyl betaine was added to the container. The contents of the container were then allowed to cool. Once at room temperature, the phenoxyethanol and methylisothiazolinone preservative and the 50 wt % solids aqueous emulsion of dimethiconol and TEA-dodecylbenzenesulfonate were added to the container. The final pH of the product shampoo formulation was then adjusted to a pH of 5 using sodium hydroxide or citric acid as necessary and sufficient water was added to adjust the total formulation weight to 100 g. Additional PEG-150 pentaerythrityl tetrastearate was added to adjust the final formulation viscosity to 11,000 cP Brookfield viscosity measured using a number 6 spindle at 30 rpm under laboratory conditions.

TABLE 4

| | CF1 | CF2 | CF3 | CF4 | CF5 | CF6 | CF7 | F1 | F2 | F3 | F4 |
|---|---|---|---|---|---|---|---|---|---|---|---|
| Ingredient INCI name | wt % active | | | | | | | | | | |
| Deionized water | q.s. 100 | | | | | | | | | | |
| Sodium lauryl ether sulfate¹ | 9 | 9 | 9 | 9 | 9 | 9 | 9 | 9 | 9 | 9 | 9 |
| Guar hydroxypropyltrimonium chloride² | 0.3 | 0 | 0 | 0 | 0 | 0 | 0 | 0 | 0 | 0 | 0 |
| Polymer from Synthesis S1 | 0 | 0.3 | 0 | 0 | 0 | 0 | 0 | 0 | 0 | 0 | 0 |
| Polymer from Synthesis S2 | 0 | 0 | 0.3 | 0 | 0 | 0 | 0 | 0 | 0 | 0 | 0 |
| Polymer from Synthesis S3 | 0 | 0 | 0 | 0.3 | 0 | 0 | 0 | 0 | 0 | 0 | 0 |
| Polymer from Synthesis S4 | 0 | 0 | 0 | 0 | 0.3 | 0 | 0 | 0 | 0 | 0 | 0 |
| Polymer from Synthesis S5 | 0 | 0 | 0 | 0 | 0 | 0.3 | 0 | 0 | 0 | 0 | 0 |
| Polymer from Synthesis S6 | 0 | 0 | 0 | 0 | 0 | 0 | 0.3 | 0 | 0 | 0 | 0 |
| Polymer from Synthesis S7 | 0 | 0 | 0 | 0 | 0 | 0 | 0 | 0.3 | 0 | 0 | 0 |
| Polymer from Synthesis S8 | 0 | 0 | 0 | 0 | 0 | 0 | 0 | 0 | 0.3 | 0 | 0 |
| Polymer from Synthesis S9 | 0 | 0 | 0 | 0 | 0 | 0 | 0 | 0 | 0 | 0.3 | 0 |
| Polymer from Synthesis S10 | 0 | 0 | 0 | 0 | 0 | 0 | 0 | 0 | 0 | 0 | 0.3 |
| Tetrasodium EDTA³ | 0.2 | 0.2 | 0.2 | 0.2 | 0.2 | 0.2 | 0.2 | 0.2 | 0.2 | 0.2 | 0.2 |
| Cocamide MEA⁴ | 1.0 | 1.0 | 1.0 | 1.0 | 1.0 | 1.0 | 1.0 | 1.0 | 1.0 | 1.0 | 1.0 |
| Cocamidopropyl Betaine⁵ | 2.1 | 2.1 | 2.1 | 2.1 | 2.1 | 2.1 | 2.1 | 2.1 | 2.1 | 2.1 | 2.1 |
| Phenoxyethanol and Methylisothiazolinone⁶ | 0.25 | 0.25 | 0.25 | 0.25 | 0.25 | 0.25 | 0.25 | 0.25 | 0.25 | 0.25 | 0.25 |
| Dimethiconol and TEA-dodecylbenzenesulfonate⁷ | 1.0 | 1.0 | 1.0 | 1.0 | 1.0 | 1.0 | 1.0 | 1.0 | 1.0 | 1.0 | 1.0 |

| | |
|---|---|
| PEG-150 Pentaerythrityl Tetrastearate⁸ | q.s. viscosity 11,000 cP |
| Sodium hydroxide or Citric acid | q.s. pH 5 |

¹available from Stepan Company under tradename STEOL ® CS-130
²available from Solvay Novecare under tradename Jaguar ® Excel
³available from The Dow Chemical Company under tradename Versene ™ 220
⁴available from Croda Inc. under tradename Incromide ™ CMEA
⁵available from Stepan Company under tradename AMPHOSOL ® CA
⁶preservative available from The Dow Chemical Company under tradename Neolone ™ PE
⁷available from The Dow Chemical Company under tradename DOWSIL ™ 1785 POE Emulsion
⁸available from Croda Inc. under tradename Crothix ™-PA-(MH)

### Silicone Deposition Analysis

The silicone deposition on hair from the shampoo formulations prepared according to Comparative Examples CF1-CF7 and Example F1-F4 was quantified using X-ray photoelectron spectroscopy (XPS), which gives a quantitative elemental and chemical state information from the top 10 nm of the hair sample.

Hair tresses (1 g, bleached hair available from International Hair Importers) were initially washed in a 9 wt % sodium lauryl sulfate solution and rinsed with water flowing at 0.4 L/min for 30 seconds. Following the initial wash step, the hair tresses were then washed with a shampoo formulation of Comparative Examples CF1-CF6 and Example F1-F2 by applying 0.8 g of the shampoo formulation to the hair tress and massaging in for 30 seconds on each side and then rinsing with water flowing at 0.4 L/min for 15 seconds on each side. The hair tresses were then evaluated using XPS. The XPS data were acquired from four areas per tress across a 1 cm² by 3 mm hair bundle. The instrument parameters used are provided in TABLE5. The mol % of the silicon from the shampoo formulation deposited on the hair is reported in TABLE6.

TABLE 5

| | |
|---|---|
| Instrument | K-Alpha X-ray photoelectron spectroscopy (XPS) |
| X-ray source: | Monochromatic Aluminum K-alpha |
| Analyzer Pass | 200 eV (survey spectra) |
| Energy | 20 eV (high resolution spectra) |
| Take-Off Angle | 90° |
| Auto height | on |
| Analysis Area | 400 μm oval |
| Flood Gun | on |
| Analysis Software | Casa 2.3.17 Dev. 6 3y |

TABLE 6

| Shampoo Formulation | Total CS | TKN (wt %) | Si deposition (mol %) |
|---|---|---|---|
| No polymer | 0 | 0 | 0.3 |
| Comp. Example CF1 | 0.340 | 1.668 | 5.7 |
| Comp. Example CF2 | 0.138 | 1.112 | 1.3 |
| Comp. Example CF3 | 0.192 | 1.409 | 1.1 |
| Comp. Example CF4 | 0.165 | 1.233 | 5.5 |
| Comp. Example CF5 | 0.146 | 1.112 | 1.2 |
| Comp. Example CF6 | 0.225 | 1.442 | 2.9 |
| Comp. Example CF7 | 0.163 | 1.079 | 2.2 |
| Example F1 | 0.134 | 1.329 | 6.8 |
| Example F2 | 0.199 | 1.461 | 9.0 |
| Example F3 | 0.171 | 1.594 | 10.4 |
| Example F4 | 0.146 | 1.706 | 11.5 |

We claim:

1. A formulation for the care of damaged hair, comprising:
a dermatologically acceptable vehicle;
a dermatologically acceptable silicone; and
a deposition aid polymer, wherein the deposition aid polymer is a cationic dextran polymer, comprising a dextran base polymer functionalized with quaternary ammonium groups; wherein the dextran base polymer has a weight average molecular weight of 50,000 to 3,000,000 Daltons; wherein the quaternary ammonium groups include
(i) a quaternary ammonium group of formula (II) bound to a pendent oxygen on the dextran base polymer 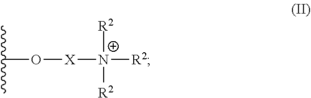
(ii) a quaternary ammonium group of formula (III) bound to a pendent oxygen on the dextran base polymer 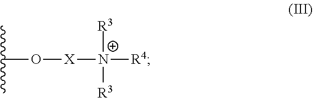
wherein 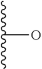 [SMILES: *O]
is a pendent oxygen on the dextran base polymer; wherein X is a divalent linking group; wherein each R² is independently selected from a C₁₋₄ alkyl group; wherein each R³ is independently selected from a C₁₋₄ alkyl group; and wherein each R⁴ is independently selected from a linear or branched C₅₋₂₀ alkyl group.

2. The formulation of claim 1, wherein the cationic dextran polymer has a Kjeldahl nitrogen content corrected for ash and volatiles, TKN, of 0.5 to 5.0 wt %.

3. The formulation of claim 2, wherein the formulation is selected from the group consisting of a leave on conditioner, a rinse off conditioner and a conditioning shampoo.

4. The formulation of claim 3, further comprising: a cleaning surfactant; wherein the formulation is a conditioning shampoo.

5. The conditioning shampoo of claim 4,
wherein the quaternary ammonium groups of formula (II) are of formula (IIa) 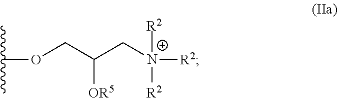
wherein the quaternary ammonium group of formula (III) are of formula (IIIa) 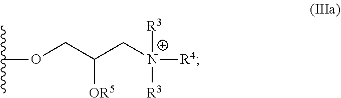
wherein each R⁵ is independently selected from the group consisting of a hydrogen and a linear or branched C₁₋₄ alkyl group.

6. The conditioning shampoo of claim 5, wherein each R² and R³ is a methyl group; and wherein each R⁵ is a hydrogen.

7. The conditioning shampoo of claim 6, wherein each R is a linear or branched C₁₂ alkyl group.

8. The conditioning shampoo of claim 7, wherein the deposition aid polymer has a Kjeldahl nitrogen content corrected for ash and volatiles, TKN, of >1 to 5.0 wt %; and wherein the deposition aid polymer has a cationic degree of substitution, DS, of dimethyldodecyl ammonium moieties of >0 to 0.03.

9. The conditioning shampoo of claim 8, further comprising at least one additional ingredient selected from the group consisting of an antimicrobial agent/preservative; a rheology modifier; a soap; a colorant; pH adjusting agent; an antioxidant; a humectant; a wax; a foaming agent; an emulsifying agent; a colorant; a fragrance; a chelating agent; a preservative; a bleaching agent; a lubricating agent; a sensory modifier; a sunscreen additive; a vitamin; a protein/amino acid; a plant extract; a natural ingredient; a bioactive agent; an anti-aging agent; a pigment; an acid; a penetrant; an anti-static agent; an anti-frizz agent; an antidandruff agent; a hair waving/straightening agent: a hair styling agent; a hair oil; natural oils or ester emollients; an absorbent; a hard particle; a soft particle; a conditioning agent; a slip agent; an opacifier; a pearlizing agent and a salt.

10. A method of caring for damaged hair, comprising:
selecting a formulation according to claim 1; and
applying the formulation to damaged hair;
wherein the deposition aid polymer enhances the deposition of the dermatologically acceptable silicone from the formulation onto the damaged hair relative to an otherwise identical formulation without the deposition aid polymer.
